(19) United States
(12) Patent Application Publication
(10) Pub. No.: US 2023/0149605 A1
(43) Pub. Date: May 18, 2023

(54) NANOBARRIER AND METHOD OF REGULATING MACROPHAGE ADHESION AND POLARIZATION USING THE SAME

(71) Applicants: Korea University Research and Business Foundation, Seoul (KR); Industry-University Cooperation Foundation Hanyang University ERICA Campus, Ansan (KR)

(72) Inventors: Heemin KANG, Seoul (KR); Do-Kyoon KIM, Ansan-si (KR); Yu-Ri KIM, Seoul (KR); Gun-Hyu BAE, Hwaseong-si (KR)

(73) Assignees: Korea University Research and Business Foundation, Seoul (KR); Industry-University Cooperation Foundation Hanyang University ERICA Campus, Ansan (KR)

(21) Appl. No.: 17/963,601

(22) Filed: Oct. 11, 2022

(30) Foreign Application Priority Data
Nov. 16, 2021 (KR) 10-2021-0157180

(51) Int. Cl. A61L 31/04; A61L 31/02; A61L 31/10

(52) U.S. Cl. CPC A61L 31/047; A61L 31/022; A61L 31/10; A61L 2400/12; A61L 2300/40

(57) ABSTRACT

The present invention relates to a nanobarrier for regulating macrophage adhesion and polarization. Moreover, the present invention relates to a method of regulating macrophage adhesion and polarization using the nanobarrier. According to the nanobarrier of the present invention and the method of regulating macrophage adhesion and polarization using the same, it is possible to efficiently regulate macrophage adhesion and polarization by applying a magnetic field to the nanobarrier.

### CROSS-REFERENCE TO RELATED APPLICATIONS

This application claims priority to and the benefit of Korean Patent Application No. 10-2021-0157180 filed in the Korean Intellectual Property Office on Nov. 16, 2021, the entire contents of which are incorporated herein by reference.

### BACKGROUND OF THE INVENTION

### Field of the Invention

The present invention relates to a magnetic nanobarrier, and more particularly, to a magnetic nanobarrier and a method of regulating macrophage adhesion and polarization using the same.

### Description of the Prior Art

Physical screens, which occur in the extracellular matrix (ECM), separate various tissue compartments to help modulate homeostasis and tissue regeneration by controlling biomolecular transport and cellular infiltration. Certain tissues can act as physical screens to modulate tissue repair mechanisms that involve the interactions of diverse cells. However, ECM-mimicking artificial materials that can dispersively and dynamically control bioactive surfaces are rare.

Integrin dynamically forms links with the bioactive-ligand-displaying-ECM, of which the RGD ligands mediate focal adhesion and intracellular mechanotransduction of cells. Remote manipulation of unscreening the ligands by light or magnetic fields can dynamically modulate cell adhesion. Conventionally, light such as ultraviolet (UV), visible, and near infrared (NIR) light has been used for photochemical manipulation of screening and unscreening of the ligands. For example, UV light has been applied to chemically cleave photosensitive polyethylene glycol-based brushes to unscreen ligand-grafted gold nanoparticles for facilitating cell adhesion. Using photoisomers such as azobenzene derivatives, screening and unscreening of the ligands via self-assembled brushes can be manipulated by illuminating UV light and visible light or single wavelengths having the ability to stimulate intracellular mechanotransduction. However, manipulation of screening and unscreening of the ligands by light in vivo has rarely been reported.

In addition, magnetic field can easily penetrate tissues in vivo to enable noninvasive control of physical screens. For example, cell adhesion can be remotely controlled by controlling screening and unscreening of the ligands through modulation of nanoparticles having magnetic properties.

### PRIOR ART DOCUMENTS

### Patent Documents

Korean Patent Application Publication No. 10-2004-0015234

### SUMMARY OF THE INVENTION

An object of the present invention is to provide a magnetic nanobarrier for regulating macrophage adhesion and polarization.

Another object of the present invention is to provide a method of regulating macrophage adhesion and polarization using a magnetic nanobarrier.

According to one aspect of the present invention, embodiments of the present invention include a nanobarrier for regulating macrophage adhesion and polarization.

The nanobarrier may comprise: magnetic barriers each comprising an aggregate of one or more magnetic particle units; a linker connected to one side of each of the magnetic barriers; and a substrate connected to the magnetic barriers via the linkers, wherein the substrate comprises ligands to which macrophages adhere.

The average diameter of the magnetic barriers may include any one or more of a first average diameter, a second average diameter, and a third average diameter, wherein the first average diameter is 150 to 250 nm, the second average diameter is 450 to 530 nm, and the third average diameter is 650 to 750 nm.

The surface of each magnetic barrier, which faces the substrate, may be spaced apart from each ligand present on the substrate by a distance of a nanogap, and the nanogap may be reversibly changed by application of a magnetic field.

The average diameter of the magnetic barriers may include the first average diameter, and the macrophage adhesion and polarization may be regulated by elongating the linker and increasing the nanogap, through pulling of the magnetic barriers in a direction away from the substrate by application of the magnetic field.

The average diameter of the magnetic barriers may include the third average diameter, the macrophage adhesion and polarization may be regulated by compressing the linker and reducing the nanogap, through pulling of the magnetic barriers in a direction toward the substrate by application of the magnetic field.

The linker may comprise: a polyethylene glycol (PEG) portion; a first bonding portion which forms a chemical bond with the magnetic barrier; and a second bonding portion which forms a chemical bond with the substrate.

The magnetic barriers in the nanobarrier may include a carboxylate group (—COO⁻), the first bonding portion may include any one of an amino group (—NH₂) and a thiol group (—SH) and form a chemical bond with the carboxylate group of the magnetic barrier, and the second bonding portion may include any one of a maleimide group and an alkenyl group (—C═C—) and form a chemical bond with a thiol group (—SH) provided on the substrate.

The linker may have a structure of the following Formula 1:

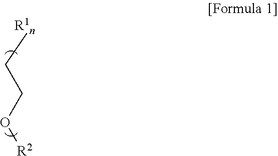

wherein R¹ may be any one of an amino group (—NH₂) and a thiol group (—SH), and R² may be any one of a maleimide group and an alkenyl group (—C═C—).

n in Formula 1 above may be 30 to 5,000.

The linker may have a length of 10 nm to 1 μm.

The ligands provided on the substrate in the nanobarrier may be bound to the surfaces of gold nanoparticles bound to the substrate.

The gold nanoparticles may be provided on the substrate by chemical bonding with a portion of the thiol groups (—SH) provided on the substrate, the ligands may be bound to the gold nanoparticles, and the linkers may be connected to the substrate by chemical bonding with the other portion of the thiol groups (—SH) provided on the substrate.

The gold nanoparticles may cover 0.001% to 10% of the area of the substrate.

70 to 85% of the area of the substrate may be covered by the magnetic barriers.

In addition, the nanobarrier according to one embodiment of the present invention may be prepared by: forming aggregates of one or more magnetic particle units; forming a carboxylate group on the surfaces of the aggregates to form magnetic barriers; binding each of the magnetic barriers to one end of each linker by stirring the magnetic barriers and the linkers; chemically binding the other end of each linker to thiol groups present on a substrate on which thiol groups and ligands are present; and deactivating thiol groups on the substrate, which remain unbound to the linkers.

The substrate may comprise a glass substrate, and thiol groups and ligands provided on at least one surface of the glass substrate, the thiol groups may be provided by thiolizing the glass substrate, at least a portion of the thiol groups may be bound to gold nanoparticles, and the ligands may be bound to the gold nanoparticles bound to the thiol groups.

Another embodiment of the present invention may include a method of regulating macrophage adhesion and polarization using the nanobarrier.

The magnetic field may be applied from outside the body to remotely control the nanobarrier in the body.

The magnetic field may have a strength of 100 mT to 500 mT.

The magnetic barriers may be pulled in a direction away from the substrate by the magnetic field to elongate the linker, thereby inhibiting macrophage M1 polarization and promoting macrophage M2 polarization.

The magnetic barriers may be pulled in a direction toward the substrate by the magnetic field to compress the linker, thereby inhibiting macrophage M2 polarization and promoting macrophage M1 polarization.

According to the present invention, it is possible to provide a magnetic nanobarrier capable of regulating macrophage adhesion and polarization.

In addition, according the present invention, it is possible to regulate macrophage adhesion and polarization using the magnetic nanobarrier.

### BRIEF DESCRIPTION OF THE DRAWINGS

a and b of FIG. 1 schematically show the process of regulating macrophage adhesion and polarization using a nanobarrier according to one embodiment of the present invention.

FIG. 2 schematically shows a process of preparing magnetic barriers according to one embodiment of the present invention.

FIG. 3 schematically shows a process of preparing magnetic barriers according to one embodiment of the present invention.

a and b of FIG. 4 show HAADF-STEM and FFT images and elemental map of magnetic barriers according to one embodiment of the present invention, and the inverse spinel structure of the Fe₃O₄ phase.

a to c of FIG. 5 show the results of measuring the SAD, EELS, and zeta potential of magnetic barriers according to one embodiment of the present invention.

a to c of FIG. 6 show the results of measuring TEM, DLS and VSM according to one embodiment of the present invention.

a to f of FIG. 7 show a substrate and gold nanoparticles on the substrate according to one embodiment of the present invention, a nanobarrier of each example, and graphs showing the results of analysis of the nanobarriers.

a and b FIG. 8; a and b of FIG. 9; a to d of FIG. 10; FIG. 11; a to e of FIG. 12; and a and b of 13 show the results of testing whether macrophage adhesion can be regulated using the nanobarrier according to one embodiment of the present invention.

a to c of FIG. 14; FIG. 15; a and b of FIG. 16; FIG. 17; and FIG. 18 show the results of experiments conducted to examine whether macrophage adhesion and polarization can be regulated using the nanobarrier according to an embodiment of the present invention.

a to c of FIG. 19; a and b of FIG. 20; and FIG. 21 show the results of experiments conducted to examine whether macrophage adhesion and polarization in vivo can be regulated, after implanting the nanobarrier according to one embodiment of the present invention into mice.

### DETAILED DESCRIPTION OF THE INVENTION

The details of other embodiments are included in the detailed description and the accompanying drawings.

The advantages and features of the present invention, and the way of attaining them, will become apparent with reference to the embodiments described below in conjunction with the accompanying drawings. However, the present invention is not limited to the embodiments disclosed below and may be embodied in a variety of different forms. Since all numbers, values and/or expressions referring to quantities of components, reaction conditions, etc., used in the present specification, are subject to the various uncertainties of measurement encountered in obtaining such values, unless otherwise indicated, all are to be understood as modified in all instances by the term “about.” Where a numerical range is disclosed herein, such a range is continuous, inclusive of both the minimum and maximum values of the range as well as every value between such minimum and maximum values, unless otherwise indicated. Still further, where such a range refers to integers, every integer between the minimum and maximum values of such a range is included, unless otherwise indicated.

In the present specification, where a range is stated for a parameter, it will be understood that the parameter includes all values within the stated range, inclusive of the stated endpoints of the range. For example, a range of 5 to 10 will be understood to include the values 5, 6, 7, 8, 9, and 10, as well as any sub-range such as 6 to 10, 7 to 10, 6 to 9, and 7 to 9, and also include any value and range between the integers which are reasonable in the context of the range stated, such as 5.5, 6.5, 7.5, 5.5 to 8.5 and 6.5 to 9. For example, a range of “10% to 30%” will be understood to include the values 10%, 11%, 12%, 13%, etc., and all integers up to and including 30%, as well as any sub-range such as 10% to 15%, 12% to 18%, 20% to 30%, etc., and also include any value between the integers which are reasonable in the context of the range stated, such as 10.5%, 15.5%, 25.5%, etc.

a and b of FIG. 1 schematically show nanobarriers having magnetic barriers of different sizes according to one embodiment of the present invention and a process of regulating macrophage adhesion and polarization by applying a magnetic field to the nanobarriers. Referring to a and b of FIG. 1, for example, when a nanobarrier having 200-nm-sized magnetic barriers is used, macrophage adhesion and polarization may be regulated by pulling the magnetic barriers in a direction away from the substrate by using a magnetic field. In addition, referring to a and b of FIG. 1, for example, when a nanobarrier having 700-nm-sized magnetic barriers is used, macrophage adhesion and polarization may be regulated by pulling the magnetic barriers in a direction toward the substrate by using a magnetic field.

A nanobarrier according to one embodiment of the present invention may comprise: magnetic barriers each comprising an aggregate of one or more magnetic particle units; a linker connected to one side of each of the magnetic barriers; and a substrate connected to the magnetic barriers via the linkers, wherein the substrate comprises ligands to which macrophages adhere.

The average diameter of the magnetic barriers may include any one or more of a first average diameter, a second average diameter, and a third average diameter. The first average diameter may be 150 to 250 nm, the second average diameter may be 450 to 530 nm, and the third average diameter may be 650 to 750 nm. Preferably, the first average diameter may be 170 nm to 230 nm, the second average diameter may be 480 nm to 520 nm, and the third average diameter may be 670 nm to 730 nm. This average diameter of the magnetic barriers may correspond to an optimal size for regulating macrophage adhesion and polarization using the barrier.

Each of the magnetic barriers may comprise an aggregate of one or more magnetic units. As the magnetic particle units, a magnetic material may be used without limitation. Preferably, the magnetic particle units may be formed of Fe₃O₄. In addition, the magnetic barriers may include, on the surface thereof, a carboxylate group (—COOH⁻) that may be bound to the linker via a chemical bond.

The magnetic barrier has a slightly polyhedral shape as shown in a and b of FIG. 1, but it may be formed in an almost spherical shape so that the inner angle formed by each face is large and the magnetic barrier has a sufficiently large number of faces so as to be close to a spherical shape. M1 polarization may be induced by restricting the binding of integrins in macrophages to the ligands present under the spherical magnetic barriers by the magnetic barriers, and conversely, M2 polarization may be induced by allowing the integrins to abundantly bind to the ligands present under the magnetic barriers.

The linker may comprise: a polyethylene glycol (PEG) portion; a first bonding portion which forms a chemical bond with the magnetic barrier; and a second bonding portion forming a chemical bond with the substrate.

The polyethylene glycol portion may be composed of a polyethylene glycol polymer and may be in the form of a long chain. The polyethylene glycol portion may be elongated or compressed depending on the direction in which the magnetic field is applied to the nanobarrier, compared to when no magnetic field is applied. Due to the nature of the polyethylene glycol portion, the linker may be elastic. Thus, in the process of regulating macrophage adhesion and polarization using the nanobarrier, the polyethylene glycol portion may be elongated or compressed depending on the direction of application of the magnetic field, thereby regulating macrophage adhesion and polarization. The weight-average molecular weight (Mw) of the polyethylene glycol portion may be 1,300 Da to 132,000 Da, preferably 3,900 Da to 132,000 Da, or 3,900 Da to 6,600 Da.

The first bonding portion may be connected to the magnetic barrier by chemical bonding with the carboxylate group (—COO⁻) of the magnetic barrier. The first bonding portion may include any one of an amino group (—NH₂) and a thiol group (—SH), but is not limited thereto and may include any one of functional groups capable of chemical bonding with the carboxylate group.

The second bonding portion may be connected to the substrate by chemical bonding with a thiol group (—SH) provided on the substrate. The second bonding portion may include any one or more of a maleimide group and an alkenyl group, but is not limited thereto and may include any one of functional groups capable of chemical bonding with the thiol group.

The linker may specifically have a structure of Formula 1 below. n in Formula 1 may be 30 to 5,000, preferably 90 to 5,000, or 90 to 150.

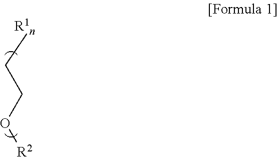

wherein R¹ may be any one of an amino group (—NH₂) and a thiol group (—SH), and R² may be any one of a maleimide group and an alkenyl group.

Preferably, the linker may be represented by Formula 2 below. n in Formula 2 may be 30 to 5,000, preferably 90 to 5,000, or 90 to 150.

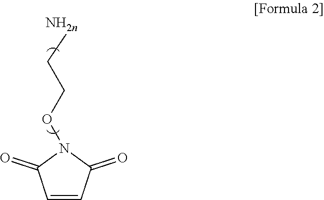

The maximum length of the linker may be such a length that, when the linkers are elongated, entanglement between the linkers may not occur or the adhesion of macrophages to the ligands may not be interfered, and when the linkers are compressed, adhesion of macrophages to the ligands may be sufficiently blocked. In addition, the minimum length of the linker may be such a length that, when the linker is elongated, a minimum nanogap size may be formed so that macrophages may adhere to the ligands, and when the linker is compressed, the ligands may be blocked by the magnetic barriers so that macrophages may not bind to the ligands. The length of the linker may be 10 nm to 1 μm, preferably 30 nm to 1 μm, or 30 nm to 50 nm.

One or more linkers may be bound to the magnetic barrier.

At least one surface of the substrate may include a ligand.

The substrate may be formed by forming thiol groups on at least one surface of a substrate, chemically bonding a portion of the thiol groups to gold nanoparticles, and coupling ligands to the gold nanoparticles. At least a portion of the thiol groups formed on the substrate may be chemically bonded to the gold nanoparticles, and ligands may not be bound to the thiol groups that have not been chemically bonded to the gold nanoparticles. The ligands may be RGD ligands.

At least a portion of the thiol groups on the glass substrate, which have not been chemically bonded to the gold nanoparticles, may be chemically bonded to the second bonding portions of the linkers.

The gold nanoparticles may be bound to the thiol groups while having a uniform distribution on the substrate. The gold nanoparticles may be added to cover 0.001% to 10% of the area of the substrate. Accordingly, the ligands bound to the gold nanoparticles may have a uniform distribution on the substrate. The gold nanoparticles may be added to cover 0.001% to 10% of the area of the substrate.

The magnetic barriers, the linkers and the substrate may be connected to one another via chemical bonds to form the nanobarrier. The nanobarrier may be formed in a linear structure.

A surface of the magnetic barrier, which faces the substrate, may be spaced apart from the ligand present on the substrate by a distance of a nanogap.

The nanogap may be reversibly changed by application of a magnetic field. The magnetic barrier may move depending on the direction of application of the magnetic field, and the nanogap may be changed depending on the moving direction of the magnetic barrier. When the magnetic barrier is pulled in a direction away from the substrate by application of the magnetic field, the nanogap may be increased as the linker is elongated. At this time, since the nanogap size is increased so that integrin may easily bind to the ligand on the substrate, macrophage adhesion to the ligand may be facilitated. In this case, anti-inflammatory M2 polarization of macrophages may be promoted. Conversely, when the magnetic barrier is pulled in a direction toward the substrate by application of the magnetic field, the nanogap may be reduced as the linker is compressed. At this time, the nanogap size is decreased so that the binding of integrin to the ligand on the substrate is inhibited, and the ligand is blocked by the magnetic barrier, so that macrophage adhesion to the ligand may be suppressed. In this case, inflammatory M1 polarization of macrophages may be promoted.

Preferably, when the nanobarrier comprises the magnetic barriers having the first average diameter, the area of the substrate, which is not covered by the magnetic barriers, increases, the degree of ligand dispersion increases, and the density of adhered macrophages decreases, so that M1 polarization may be achieved. In this case, when the nanogap is increased by elongating the linker through pulling of the magnetic barrier in a direction away from the substrate by application of the magnetic field to the nanobarrier, macrophages may adhere to even the ligands covered by the magnetic barriers, and M1 polarization may be inhibited. Therefore, when the nanobarrier comprising the magnetic barriers having the first average diameter is used, M1 polarization may be inhibited while M2 polarization may be promoted.

In addition, preferably, when the nanobarrier comprises the magnetic barriers having the third average diameter, the area of the substrate, which is not covered by the magnetic barriers, decreases, the degree of ligand dispersion decreases, and the density of adhered macrophages increases, so that M2 polarization may be achieved. In this case, when the nanogap is reduced by compressing the linker through pulling of the magnetic barrier in a direction toward the substrate by application of the magnetic field to the nanobarrier, the ligand may be blocked by the magnetic barrier, thereby reducing macrophage adhesion and inhibiting M2 polarization. Therefore, when the nanobarrier comprising the magnetic barriers having the third average diameter is used, M2 polarization may be inhibited while M1 polarization may be promoted.

The magnetic barriers may cover 70 to 85% of the area of the substrate. The density of the magnetic barriers on the substrate may vary depending on to the size of the magnetic barriers, but the percentage of the area of the substrate, which is covered by the magnetic barriers, may be maintained constant. Accordingly, the percentage of the area of the substrate, which is not covered by the magnetic barriers, may be constant. The maximum value at which the magnetic barriers cover the area of the substrate may be in a range in which it is possible to form a space in which macrophages can adhere to the ligands, without interference between the magnetic barriers in the process of regulating macrophage adhesion and polarization using the nanobarrier. In addition, the minimum value at which the magnetic barriers cover the area of the substrate may be in a range in which a sufficient amount of the magnetic barriers may exist so that the magnetic barriers can regulate macrophage adhesion and polarization in the process of regulating macrophage adhesion and polarization using the nanobarrier.

According to another embodiment of the present invention, the nanobarrier may be prepared by: forming aggregates of one or more magnetic particle units; forming a carboxylate group on the surfaces of the aggregates to form magnetic barriers; binding each of the magnetic barriers to one end of each linker by stirring the magnetic barriers and the linkers; chemically binding the other end of each linker to thiol groups on a substrate on which thiol groups and ligands are present; and deactivating thiol groups on the substrate, which remain unbound to the linkers.

The magnetic particle units may be magnetic particles, and the magnetic barriers may exhibit magnetism by including the magnetic particle units. The magnetic particle units may be combined together by hydrophobic interaction to form an aggregate. Specifically, a microemulsion may be prepared by suspending the magnetic particle units in chloroform and adding the suspension to a solution containing dodecyltrimethylammonium bromide (DTAB), and chloroform may be evaporated from the microemulsion, whereby an aggregate may be formed by hydrophobic interaction between the magnetic particle units. In the process in which close-packed nanoassembly of the magnetic particle units occurs, the surface of the aggregate of the magnetic particle units may be surrounded by amphiphilic DTAB, and then the aggregate may be stabilized by hydrophilic interaction with water. In this case, DTAB may surround the surface of the aggregate of the magnetic particle units to form a micelle structure. Therefore, the size of the aggregate may be adjusted by adjusting the amount of DTAB. Specifically, when the amount of DTAB is reduced, an area that may be surrounded by DTAB may be reduced, so that a large aggregate may be formed, thereby forming a magnetic barrier having a large size. Conversely, when the amount of DTAB is increased, an area that may be surrounded by DTAB may increase, so that a small aggregate may be formed, thereby forming a magnetic barrier having a small size.

The size of the aggregate may vary depending on the amount of the magnetic particle units combined together. The aggregate has a slightly polyhedral shape, but it may be formed in an almost spherical shape so that the inner angle formed by each face is large and the aggregate has a sufficiently large number of faces so to be close to a spherical shape. The first average diameter may be 150 nm to 250 nm, the second average diameter may be 450 nm to 530 nm, and the third average diameter may be 650 nm to 750 nm. Preferably, the first average diameter may be 170 nm to 230 nm, the second average diameter may be 480 nm to 520 nm, and the third average diameter may be 670 nm to 730 nm. This average diameter of the magnetic barriers may correspond to an optimal size for regulating macrophage adhesion and polarization using the nanobarrier.

The magnetic barrier may be formed by providing a carboxylate group on the surface of the aggregate. The carboxylate group may be formed by adding ethylene glycol containing a polyanion to a solution containing the aggregates. The polyanion may be polyacrylic acid (PAA).

The linker may comprise: a polyethylene glycol (PEG) portion, a first bonding portion, and a second bonding portion.

One end of the linker, which is chemically bonded to the magnetic barrier, may be the first bonding portion. The first bonding portion may include any one functional group selected from among an amino group and a thiol group, and may be connected to the carboxylate group of the magnetic barrier by chemical bonding. The chemical bonding between the first bonding portion and the carboxylate group of the magnetic barrier may be performed by mixing and stirring a solution containing the linkers and a solution containing the magnetic barriers.

In addition, the other end of the linker, which is chemically bonded to the substrate, may be the second bonding portion. Here, thiol groups and ligand-bearing gold nanoparticles may be present on at least one surface of the substrate. The second bonding portion may include any one of a maleimide group and an alkenyl group (—C═C—), and may be connected to the thiol group on the substrate by chemical bonding. The chemical bonding between the second bonding portion and the substrate may be performed through a thiol-ene reaction.

After the magnetic barriers, the linkers and the substrate are combined together, there may be unreacted thiol groups on the substrate, and the unreacted thiol groups may be deactivated. The deactivated thiol groups no longer react with the linkers, gold nanoparticles and ligands, and macrophages may not adhere thereto. The unreacted thiol groups may be deactivated by a compound such as the following Formula 3.

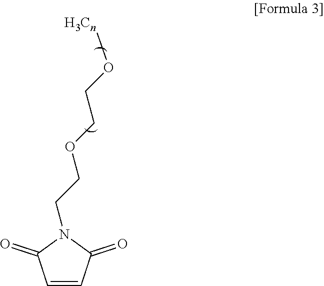

wherein n may be 10 to 5,000, preferably 10 to 30. The maleimide group in Formula 3 above may be chemically bonded to the unreacted thiol groups on the substrate through a thiol-ene reaction. This deactivation of the substrate makes it possible to block/unblock the ligands from macrophage adhesion using the magnetic barriers.

The substrate may comprise a glass substrate, and thiol groups and ligands provided on at least one surface of the glass substrate. Here, the thiol groups may be provided by thiolating the glass substrate with mercaptopropylsilatran, and at least a portion of the thiol groups may be bound to gold nanoparticles, and the ligands may be bound to the gold nanoparticles bound to the thiol groups. A compound which is used to provide the thiol groups is not limited to mercaptopropylsilatran, and any compound capable of providing thiol groups on the glass substrate may be used without limitation.

The thiol groups may be formed by thiolating the glass substrate with mercaptopropylsilatran after activating the glass substrate with sulfuric acid. At least a portion of the thiol groups may be bonded to gold nanoparticles by Au—S bonding by incubation in a solution containing the gold nanoparticles. At least a portion of the thiol groups may be bonded to gold nanoparticles by Au—S bonding by treating the glass substrate with a solution containing citrate-capped gold nanoparticles. The gold nanoparticles may be bound to at least a portion of the thiol groups, but not all of the thiol groups. Thereafter, the substrate having the gold nanoparticles bound thereto may be treated with a solution containing a thiolated RGD tripeptide (CDDRGD), so that the ligands may be bound to the gold nanoparticles. The ligands may be RGD ligands.

Another embodiment of the present invention may include a method of regulating macrophage adhesion and polarization using a nanobarrier. Here, the nanobarrier may be the nanobarrier according to the embodiment described above.

The method of regulating macrophage adhesion and polarization may comprise regulating macrophage adhesion and polarization by applying a magnetic field to the barrier. Specifically, the magnetic barriers in the nanobarrier are magnetic and thus may be attracted in the direction in which the magnetic field is applied, and the nanogap may change depending on the direction in which the magnetic field is applied, so that macrophage adhesion and polarization may be regulated. Here, the nanogap may be a gap between the lower surface of the magnetic barrier and the ligand when the substrate is assumed to be the bottom. When the magnetic barriers are pulled in a direction away from the substrate by application of the magnetic field, the number of ligands capable of binding to integrins of macrophages may increase and the density of adhered integrins may increase, thereby promoting M2 polarization and inhibiting M1 polarization. Conversely, when the magnetic barriers are pulled in a direction toward the substrate by application of the magnetic field, the number of ligands capable of binding to integrins of macrophages may decrease and the density of adhered integrins may decrease, so that M1 polarization may be promoted and M2 polarization may be inhibited.

The magnetic field may be applied from outside the body to remotely control the nanobarrier in the body, thereby regulating macrophage adhesion and polarization.

The magnetic field may be applied at a strength of 100 mT to 500 mT.

Preferably, when the nanobarrier comprises the magnetic barriers having the first average diameter, the degree of ligand dispersion on the substrate may increase and the density of adhered macrophages may decrease, so that M1 polarization may be achieved. In this case, when the nanogap is increased by elongating the linker through pulling of the magnetic barriers in a direction away from the substrate by application of a magnetic field to the nanobarrier, macrophages may adhere even to the ligands covered by the magnetic barriers and adhesion of the macrophages may increase, so that M1 polarization may be inhibited while macrophage M2 polarization may be promoted. Here, the first average diameter may be 150 nm to 250 nm, preferably 170 nm to 230 nm.

In addition, preferably, when the nanobarrier comprises the magnetic barriers having the third average diameter, the degree of ligand dispersion on the substrate may decrease and the density of adhered macrophages may increase, so that M2 polarization may be achieved. In this case, when the nanogap is reduced by compressing the linker through pulling of the magnetic barriers in a direction toward the substrate by application of the magnetic field to the nanobarrier, the ligands may be blocked by the magnetic barriers and adhesion of the macrophages may decrease, so that M2 polarization may be inhibited. In this case, macrophage M1 polarization may be promoted. Here, the third average diameter may be 650 nm to 750 nm, preferably 670 nm to 730 nm.

Hereinafter, examples of the present invention and comparative examples will be described. However, the following examples are only preferred examples of the present invention, and the scope of the present invention is not limited by the following examples.

### EXAMPLES—PREPARATION OF NANOBARRIER

### Example 1

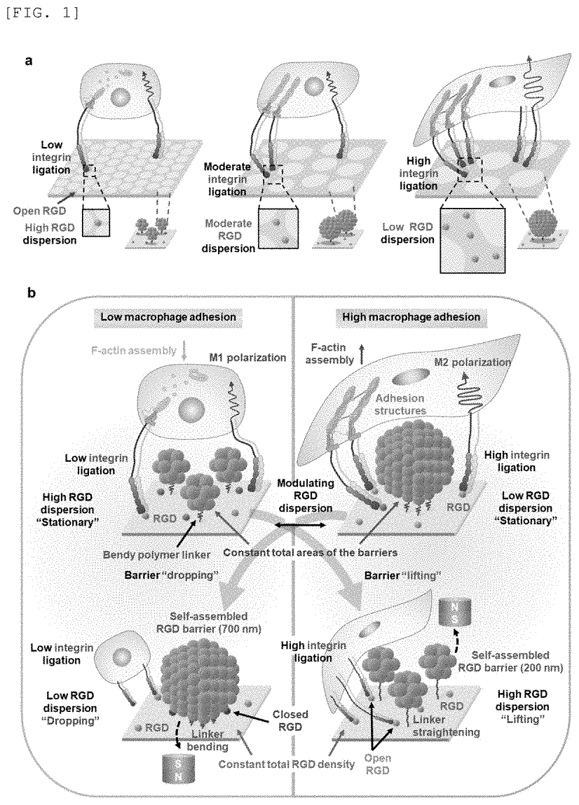

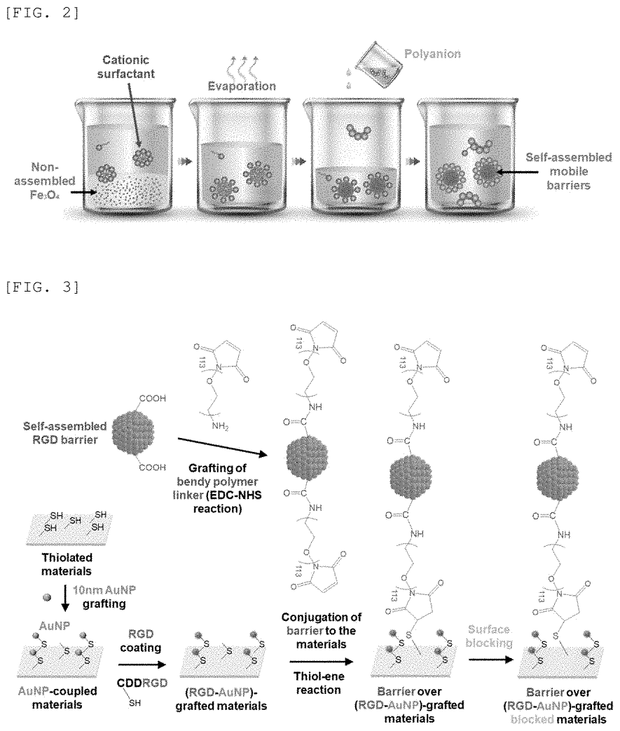

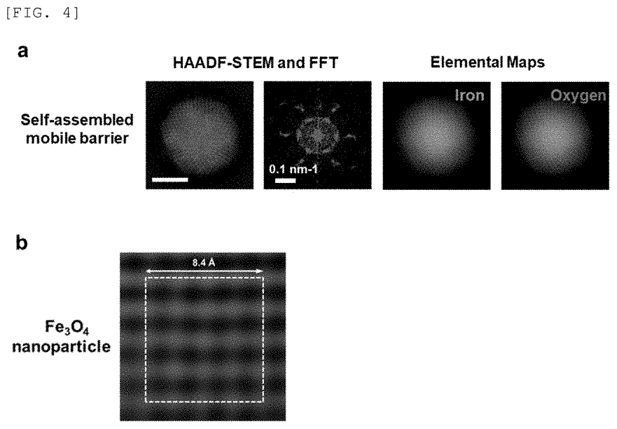

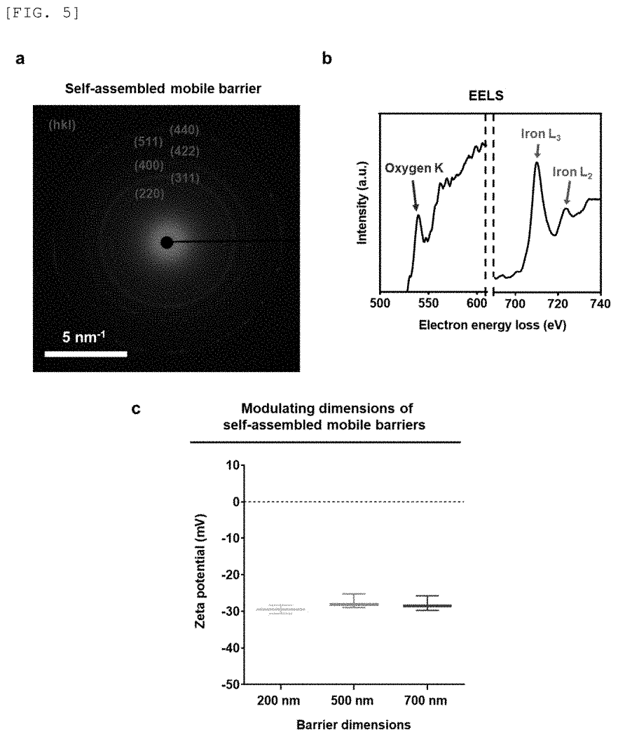

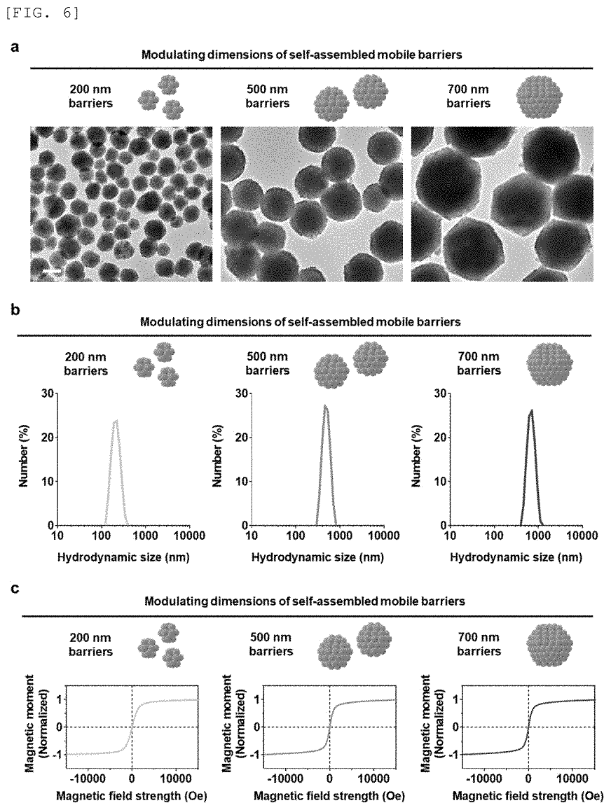

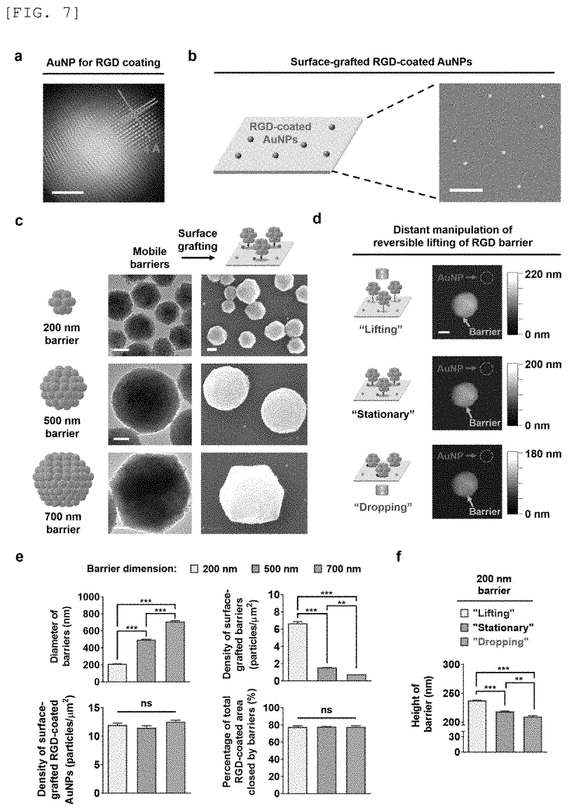

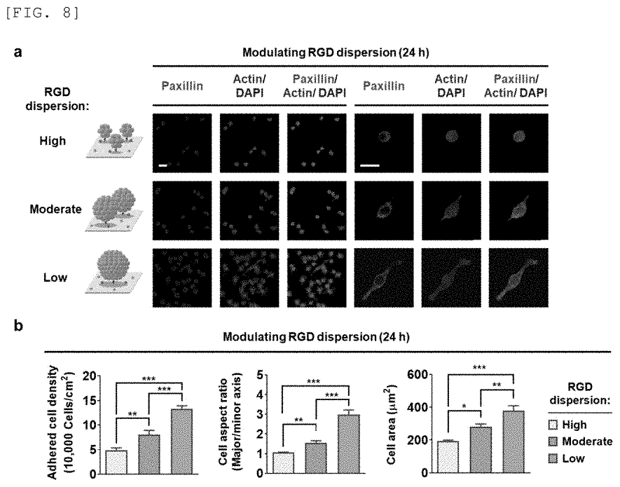

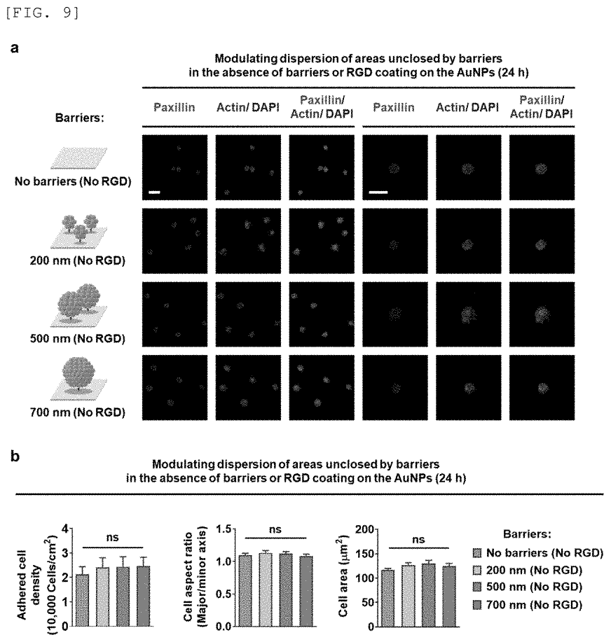

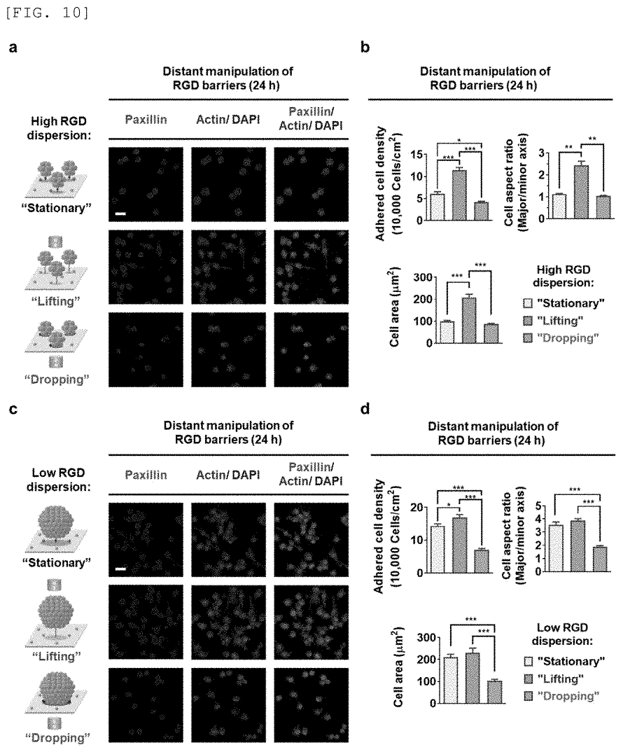

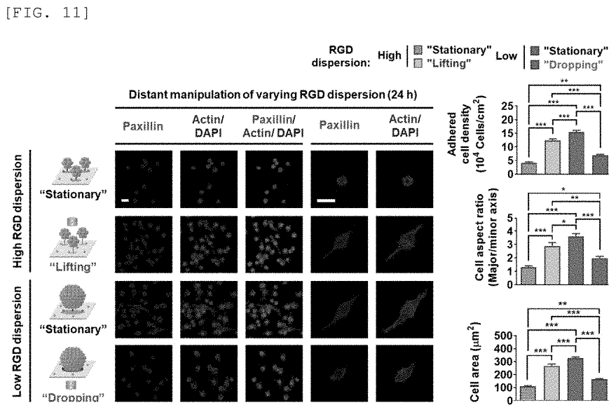

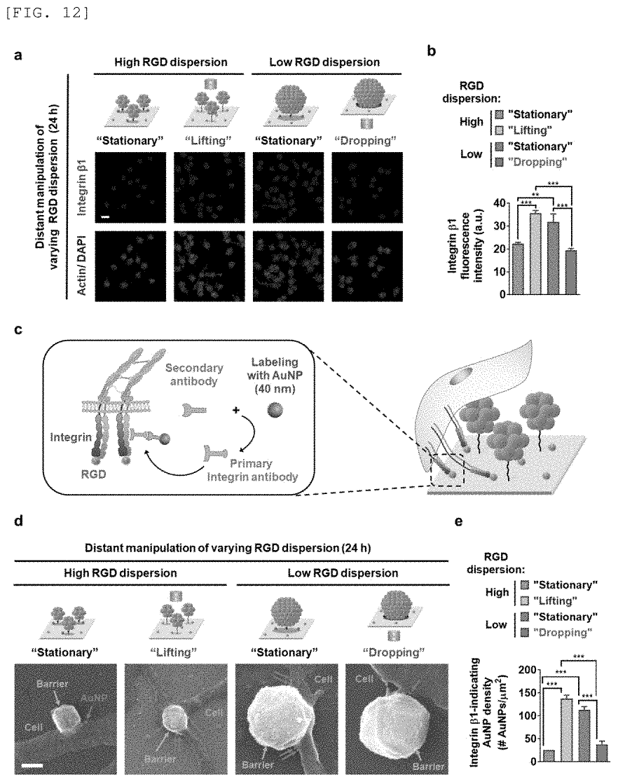

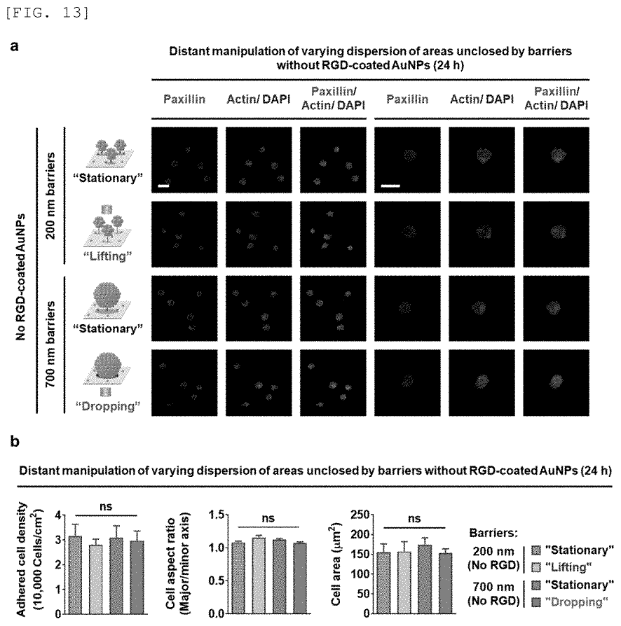

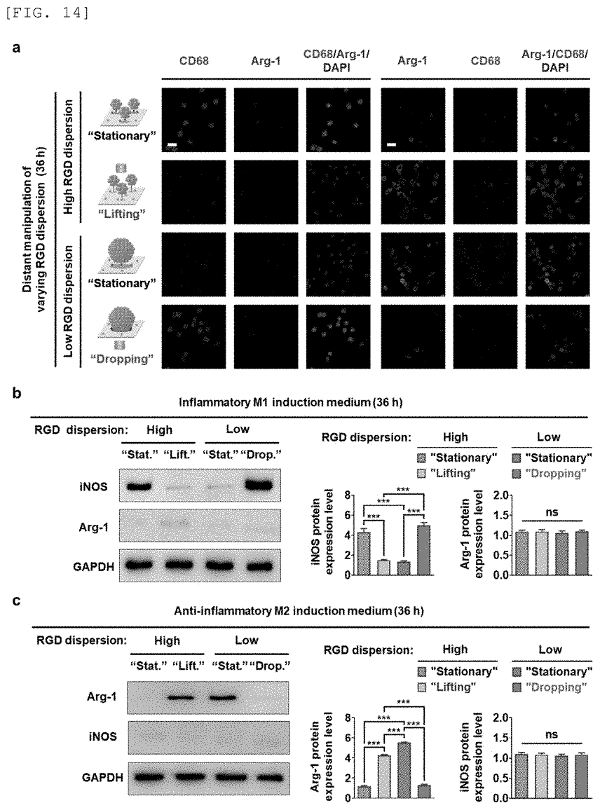

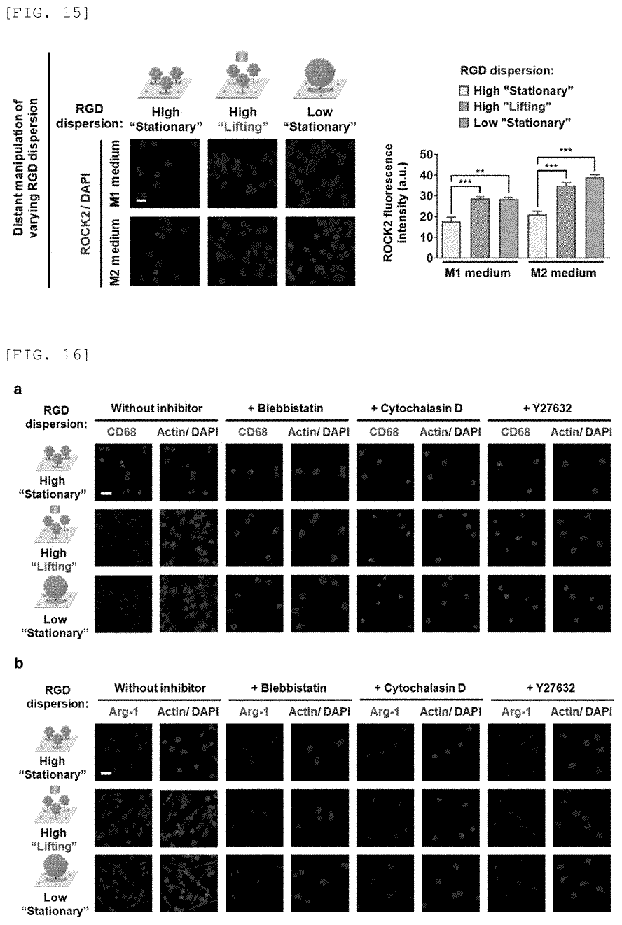

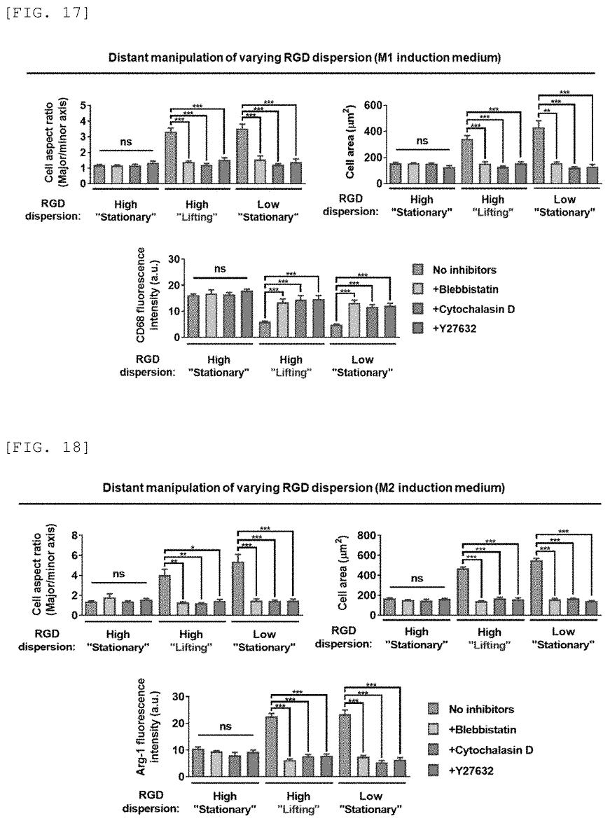

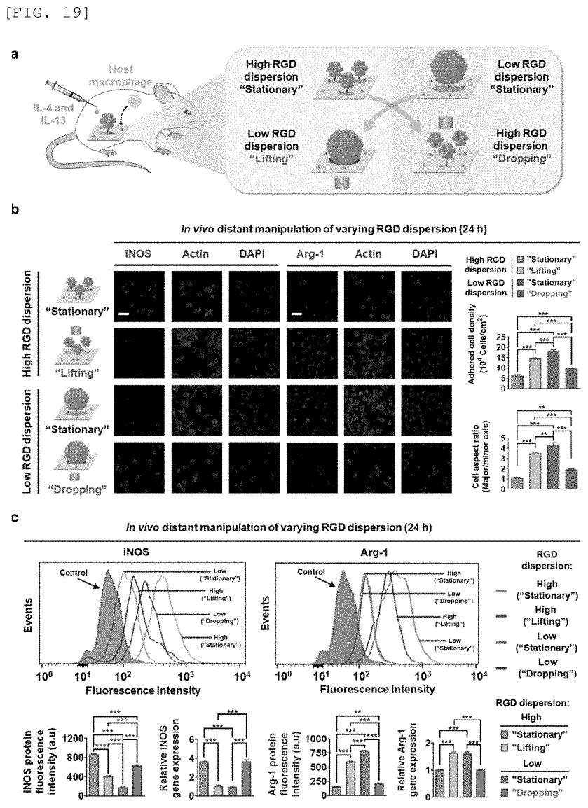

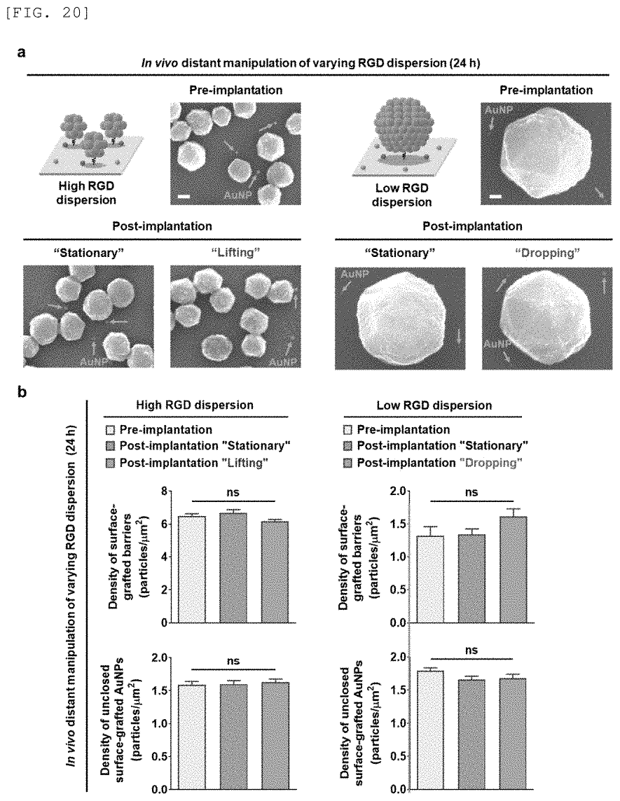

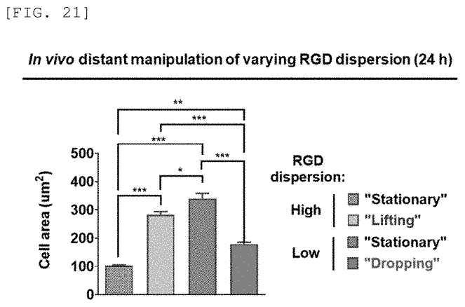

FIG. 2 schematically shows a process of preparing magnetic barriers at a test tube scale, and FIG. 3 is a schematic view showing a process of preparing a nanobarrier by preparing a substrate and grafting magnetic barriers thereto.

1. Preparation of Magnetic Barriers

Fe₃O₄ nanoparticles were used as magnetic particle units for magnetic barriers, and aggregates were formed by self-assembly of the Fe₃O₄ nanoparticles.

Fe₃O₄ nanoparticles (75 mg) were suspended in chloroform (2.3 g) and added to DI water (5 g) containing 75 mg of cationic surfactant dodecyltrimethylammonium bromide (DTAB) to create a microemulsion. The chloroform was then evaporated off by agitation at 25° C. for 16 h. During this process, self-assembly of the Fe₃O₄ nanoparticles into spherical aggregates occurred through van der Waals interactions with DTAB. The aggregates were further capped by polyanions [poly(acrylic acid) (PAA) (0.5 g)] in ethylene glycol (5.5 g) via electrostatic interactions. Finally, the polyanion-capped barriers were washed with DI water. Through this process, magnetic barriers including about 200-nm-sized aggregates were prepared.

2. Preparation of Substrate

(1) Preparation of Gold Nanoparticles

100 mL of DI water containing 1 mM gold (III) chloride trihydrate (HAuCl₄.3H₂O) was boiled at 100° C. for 20 minutes with vigorous stirring. 39 mM sodium citrate tribasic dihydrate was added to this solution and vigorously stirred for 10 minutes, thus obtaining a suspension. This suspension, which exhibited a red color due to citrate-capped gold nanoparticles (AuNPs), was cooled to 25° C., thus preparing a gold nanoparticle suspension.

(2) Thiolation of Substrate

A 1×1 cm square glass coverslip (cell culture grade) was used as a material for a substrate. The substrate surface was cleaned with a mixture of HCl and MeOH (1:1) for 30 min and then rinsed with DI water. Next, the substrate was activated with sulfuric acid for 1 h and serially rinsed with DI water and methanol. The activated substrate surface was thiolated in methanol mixed with mercaptopropylsilatrane (0.5 mM) for 1 h in the dark and then serially rinsed with methanol and DI water. The thiolated substrate surface was treated with a suspension of citrate-capped gold nanoparticles (1.7 nM) at 25° C. for 2 h. The gold nanoparticles were grafted onto the thiolated substrate surface via Au—S bonding.

(3) Binding of Ligands to Thiolated Substrate

The substrate surface presenting the gold nanoparticles was serially rinsed with sodium citrate solution and DI water, and then was treated with a solution containing DMSO mixed with thiolated RGD tripeptide (CDDRGD from GL Biochem, 0.2 nM), tris(2-carboxyethyl) phosphine hydrochloride (TCEP, 10 mM), and N, N-diisopropylethylamine (DIPEA, 0.2%) at 25° C. for 12 h in the dark, followed by rinsing with DI water. Through (1) to (3) above, the substrate presenting ligand-bearing gold nanoparticles was prepared.

3. Binding of Magnetic Barriers to Substrate

The 200-nm-sized magnetic barriers were calculated to be roughly 1.7×10¹⁰ in DI water (1 mL), which decreased with increasing magnetic barrier dimensions. The EDC/NHS reaction-based PEGylation of the magnetic barriers was performed. To this end, Mal-PEG-amine (0.4 mg, Mw: 5 kDa, Biochempeg), N,N-di-isopropyl-ethylamine (DIPEA, 0.2%), N-ethyl-N′-(3-(dimethylaminopropyl) carbodiimide (EDC, 1.4 mg), and N-hydroxy-succinimide (NHS, 6.4 mg) were added to DI water (1 mL) containing the magnetic barriers. This reaction mixture was then vigorously vortexed for 16 h in the dark and rinsed with DI water. As a result, magnetic barriers bound to PEG linkers capable of compression and elongation were prepared.

The magnetic barriers bound to the linkers were grafted onto the substrate (prepared in step 2) by the thiol-ene reaction.

Thereafter, the thiol groups not bound to the gold nanoparticles or the linkers were deactivated by treatment with 1 ml DI water containing 100 μl of methoxy-PEG-maleimide. The weight-average molecular weight (Mw) of methoxy-PEG-maleimide used herein was 2 kDa.

Through the above-described process, a nanobarrier (Example 1) including 200-nm-sized magnetic barriers was prepared.

### Examples 2 and 3

Examples 2 and 3 were prepared in the same manner as Example 1, except that the size of magnetic barriers was changed.

In Example 2, 5 g of DI water containing 50 mg of dodecyltrimethylammonium bromide (DTAB) was added to 2.3 g of chloroform during the process of forming aggregates. In addition, in the step of binding the magnetic barriers to the substrate, the magnetic barriers were added at a concentration of about 2.2×10⁹ per mL of DI water. Other preparation processes were carried out in the same manner as in Example 1.

Thereby, a nanobarrier (Example 2) including 500-nm-sized magnetic barriers was prepared.

In Example 3, 5 g of DI water containing 25 mg of dodecyltrimethylammonium bromide (DTAB) was added to 2.3 g of chloroform during the process of forming aggregates. In addition, in the step of binding the magnetic barriers to the substrate, the magnetic barriers were added at a concentration of about 0.8×10⁹ per mL of DI water. Other preparation processes were carried out in the same manner as in Example 1.

Thereby, a nanobarrier (Example 2) including 700-nm-sized magnetic barriers was prepared.

### Comparative Examples

For comparison with the effects of the Examples, nanobarriers of Comparative Examples were prepared.

The nanobarrier of Comparative Example 1 was prepared in the same manner as in Example 1, except that the magnetic barriers and the linkers were not used.

The nanobarrier of Comparative Example 2 was prepared in the same manner as in Example 1, except that the linkers were not excluded.

### EXPERIMENTAL EXAMPLES

The terms used in the description of the experimental examples and the drawings related thereto are as follows. The nanobarrier of Example 1 may be denoted as Example 1 or a nanobarrier having 200-nm-sized magnetic barriers, and may be denoted as high ligand dispersion, high RGD dispersion, or high conditions in relation to the degree of ligand dispersion. The nanobarrier of Example 2 may be denoted as Example 2 or a nanobarrier having 500-nm-sized magnetic barriers. The nanobarrier of Example 3 may be denoted as Example 3, a nanobarrier having 700-nm-sized magnetic barriers, and may be denoted as low ligand dispersion, low RGD dispersion, and low conditions in relation to the degree of ligand dispersion. In addition, the magnetic barriers of Example 1 may be denoted as 200 nm, the magnetic barriers of Example 2 may be denoted as 500 nm, and the magnetic barriers of Example 3 may be denoted as 700 nm. In addition, in relation to tuning of the nanogap, a state in which no magnetic field is applied may be denoted as stationary or stationary, a state in which the nanogap is increased may be denoted as lift, lifting, lift state, or lifting state, and a state in which the nanogap is reduced may be denoted as drop, dropping, drop state, or dropping state.

### Experimental Example 1—Analysis of Magnetic Barriers

1. High Angle Annular Dark Field-Scanning Transmission Electron Microscopy (HAADF-STEM)

Magnetic barriers of different sizes were analyzed to confirm the nanoscale self-assembly of the Fe₃O₄ nanoparticles and the angstrom-scale atomic arrangement within each Fe₃O₄ nanoparticle via HAADF-STEM imaging using a probe Cs-corrected JEM ARM200CF (JEOL Ltd.). In brief, the following measurement conditions were used: 200 kV, spherical aberration (C3) of 0.5 to 1 μm, a measured phase of 27 to 28 mrad, a convergence semi-angle of 21 mrad, a collection semi-angle of 90 to 370 mrad, electron probe sizes of 8C (1.28 Å) and 9C (1.2 Å), a pixel dwell time of 10 to 15 μs, a pixel area of 2,048×2,048, an emission current of 8 to 13 pA, and a probe current range of 10 to 20 pA. Here, the scale bar is 100 nm.

The HAADF-STEM image is shown in a of FIG. 4. Here, the scale bar is 100 nm. The image divulged that the atomic arrangement within the crystalline Fe₃O₄ nanoparticles in the barriers corresponded to the inverse spinel structure in the Fe₃O₄ phase. b of FIG. 4 shows the inverse spinel structure in the Fe₃O₄ phase.

2. Fast Fourier Transform (FFT) Analysis of Barriers

The FFT image is shown in a of FIG. 4. Here, the scale bar is 0.1 nm−1. The ordered arrangement of the aggregates in the magnetic barriers was examined by applying FFT to the HAADF-STEM images taken at low magnification and accurately aligned along the molecular (not atomic) zone axis. The hexagonal array spots in the FFT analysis proved the close packing of the aggregates in the magnetic barriers.

3. Selected Area Diffraction (SAD) Analysis of Magnetic Barriers

The SAD image is shown in a of FIG. 5. The scale bar is 5 nm. The atomic arrangement within the crystalline Fe₃O₄ nanoparticles in the magnetic barriers was examined via SAD analysis at a camera length of 6 cm. The image was indexed to the (hkl) diffraction planes of the Fe₃O₄ phase. The collected crystallographic information was displayed in the SAD pattern exhibiting multiple concentric diffraction rings, thereby proving the random orientation of the Fe₃O₄ nanoparticle. The diffraction rings represented the (220), (311), (400), (422), (511), and (440) crystallographic planes, and corresponded to interplanar d-spacings of 2.97, 2.53, 2.09, 1.72, 1.61, and 1.48 Å, respectively, which precisely matched with their reported values.

4. Energy-Dispersive X-Ray Spectroscopy-Based Elemental Maps

The elemental composition of the aggregates was analyzed via elemental maps, and the results are shown in a of FIG. 4. In this measurement, 200-nm-sized aggregates were measured, and two SDD detectors were used. As a result, it could be confirmed that iron (Fe) and oxygen (O) were elements uniformly present in each of the aggregates. This confirms that the Fe₃O₄ nanoparticles retained their elements and magnetism after their self-assembly into the magnetic barriers.

5. Electron Energy Loss Spectroscopy (EELS)

The elemental composition of iron and oxygen present in the magnetic barriers was examined via EELS analysis. In this measurement, 200-nm-sized aggregates were measured, and the results are shown in b of FIG. 5. In this measurement, 200 kV was applied using a Cs-corrected JEM ARM200CF probe (JEOL Ltd.) with a Gatan K2 summit electron detector. Dual modes including electron counting and 965 GIR Quantum ER were used to correct the edge energy calibration of the zero loss peak. As a result, the EELS fine edge structures displaying peaks corresponding to iron L₃ (710 eV) and L₂ (723 eV) and oxygen K (539 eV) could be revealed in the EELS spectra without exhibiting close overlaps in the background subtraction. From the measurement results, it could be confirmed that the aggregates retained their magnetism.

6. Zeta Potential Measurement of Magnetic Barriers

The surface charges of the magnetic barriers were analyzed, and the results are shown in c of FIG. 5. The surface charges of the magnetic barriers were analyzed by obtaining zeta potentials of the negatively charged polyanion-capped magnetic barriers at 25° C. using Zetasizer Nano ZS90 Malvern Panalytical (Malvern, UK). From the analysis results, it could be confirmed that each magnetic barrier exhibited a negative charge, exhibiting −20 mV to −40 mV. Data are presented as mean±standard error (n=3).

7. Dynamic Light Scattering (DLS) Measurement and Transmission Electron Microscopy (TEM) Analysis of Magnetic Barriers

DLS and TEM measurements were performed to measure the dimensions of the magnetic barriers, and the results are shown in a to c of FIG. 6. The distribution in the dimensions and morphology of the magnetic barriers was broadly estimated via low magnification TEM analysis conducted with a Tecnai 20 (FEI, USA). Five different acquired images in each dimension group were used for the quantification.

a of FIG. 6 shows TEM images, and the scale bar indicates 200 nm. b of FIG. 6 shows the results of DLS analysis, and data are shown as mean±standard error (n=30). From the TEM images, it can be seen that the magnetic barriers of different dimensions were formed in a polyhedral shape close to a sphere. In the DLS analysis, the dimensions were computed as 209.6±5.1 nm for the magnetic barriers prepared in Example 1, 496.2±7.4 nm for the magnetic barriers prepared in Example 2, and 708.5±18.8 nm for the magnetic barriers prepared in Example 3. The dimensions of the nanobarriers, measured by TEM and DLS, were quantified, and the results are graphed in e of FIG. 7. The experimental results prove that the dimensions intended to be prepared in each example are identical to the actual measured dimensions.

8. Vibrating Sample Magnetometry (VSM) Measurement of Magnetic Barriers

The magnetic barriers were subjected to VSM measurement to examine their magnetization properties. The mobile barriers of different dimensions were subjected to VSM measurements using an EV9 (Microsense) to examine their reversible magnetization properties. The measurements were conducted at 27° C. to obtain hysteresis loops displaying the magnetic moments as a function of applied magnetic field strength. The magnetic moments were presented after normalization to the maximum saturation magnetization value for each dimension group of the magnetic barriers. From the measurement results, it can be seen that each magnetic barriers exhibited reversible magnetization properties. Due to these reversible magnetization properties, the nanobarrier can be adjusted by applying a magnetic field.

### Experimental Example 2—Analysis of Nanobarriers

The morphologies of the nanobarriers prepared in the Examples were analyzed.

1. High Resolution-STEM (HR-STEM) Characterization of Gold Nanoparticles (AuNPs)

The gold nanoparticles prepared in Example 1 2(1) were analyzed, and the results are shown in a of FIG. 7. 10 nm gold nanoparticles were synthesized to exhibit atomic-level crystallinity. The synthesized gold nanoparticles were subjected to HR-STEM imaging to identify and label the average lattice spacing between the periodic lattice fringes. The average lattice spacing between the periodic lattice fringes was 2.4 Å, which is consistent with the previously reported value for the crystalline phase of the gold nanoparticles.

2. Analysis of Ligand Dispersion and Nanobarriers

The nanobarriers and ligand dispersion were analyzed. SEM imaging was performed using a Quanta 250 FEG scanning electron microscope (FEI). The nanobarriers were platinum-coated for SEM imaging. The nanobarriers were dried in a vacuum and platinum-coated for SEM imaging. ImageJ software was used for various computations from four different SEM images.

b of FIG. 7 shows a schematic view and SEM image of the substrate, and dots on the substrate indicate ligand-bound gold nanoparticles. Here, the scale bar indicates 200 nm. Here, the densities of the ligand-bound gold nanoparticles are displayed as particles per μm². The SEM image demonstrates the homogeneous distribution of the ligand-bound gold nanoparticles (12.0±1.4 nanoparticles per μm²). The results regarding the homogeneous distribution are graphed in e of FIG. 7.

c of FIG. 7 shows images the nanobarriers of different sizes, each comprising the magnetic barriers bound to the substrate, and shows images of the magnetic barriers and images after the magnetic barriers were bound to the substrate. In the images, AuNPs (indicated by the red arrow) are ligand-coated gold nanoparticles. The density of the magnetic barriers, which gradually declined with escalating magnetic barrier dimensions, was computed and displayed as particles per μm². The magnetic barriers were distributed almost uniformly on the substrate, and a nanogap was formed between the magnetic barrier and the ligand on the substrate. The RGD area closed by the magnetic barriers was calculated and quantified, and the results are shown in e of FIG. 7.

As the concentrations of the magnetic barriers were lowered with increasing magnetic barrier dimensions, the surface-grafted density of the magnetic barriers was gradually decreased, while maintaining a similar total area of unblocked ligand; the densities of the surface-grafted magnetic barriers were computed as 6.7±0.3, 1.6±0.1, and 0.8±0.1 magnetic barriers/μm² for 200, 500, and 700 nm magnetic barriers, respectively. The results are shown in e of FIG. 3. Although the density of the bound magnetic barriers varied depending on the size of the bound magnetic barriers, the percentages of total RGD-coated area blocked by the magnetic barriers were comparable (in the range from about 77.6% to 78.0%). Therefore, it can be confirmed that the blocked ligand area remains similar regardless of the size of the magnetic barriers.

3. Test for Linker Manipulation by Magnetic Field

It was tested whether the linker could be elongated or compressed in a contactless manner by applying a magnetic field to the nanobarrier, and the results are shown in d and f of FIG. 7. In this experiment, the nanobarrier of Example 1 was used, and the contactless manipulation of lifting and dropping of the magnetic barriers was confirmed via magnetic atomic force microscopy (AFM) imaging. An XE-100 System (Asylum Research) was exploited for in situ magnetic AFM imaging (in AC, air mode at 25° C.) using an SSS-SEIHR-20 AFM cantilever (Nanosensors) with a spring constant of 5 to 37 N/m and a resonance frequency of 96 to 175 kHz. In this experiment, when the linker was elongated by pulling the magnetic barriers in a direction away from the substrate by application of a magnetic field to the nanobarrier, the nanogap, which is a gap between the magnetic barrier and the ligand, increased, and when the linker was compressed by pulling the magnetic barriers in a direction toward the substrate by application of a magnetic field, the nanogap decreased. Specifically, the results were presented by imaging an identical area of the substrate surface three times in each of the “lifting”, “stationary”, and “dropping” states. Under the condition in which the magnetic field was applied, the magnetic field was applied at 290 mT.

Referring to the AFM images in d of FIG. 7, it can be seen that the morphology or size of the magnetic barriers did not change depending on whether the magnetic field was applied. AuNPs (indicated by red circles and arrows) are ligand-coated gold nanoparticles, which appear faint due to their small size (10 nm) and low height. By analyzing the AFM images, the distance between the upper surface of the magnetic barrier and the substrate (which was assumed to be the bottom) was measured. The measured values were 237.7±0.6 nm in “lifting”, 218.3±1.5 nm in “stationary”, and 209.7±2.5 nm in “dropping”. Although not shown in the figures, the values measured for the nanobarriers having the 500-nm and 700-nm magnetic barriers, respectively, were almost the same as the values measured for the nanobarrier having the 200-nm magnetic barriers. This means that the nanogap size can be tuned by the magnetic field, thereby effectively modulating macrophage adhesion.

### Experimental Example 3—Whether or not to Regulate Macrophage Adhesion Using Nanobarriers

Proteins expressed upon macrophage adhesion were subjected to immunofluorescent staining to determine whether macrophages adhered to the nanobarrier. After culturing macrophages, their structures were preserved by immersing them in 4% paraformaldehyde (PFA) for 12 min. After rinsing with phosphate-buffered saline (PBS), the macrophages were treated with a blocking solution containing PBS mixed with 3% bovine serum albumin and 0.1% Triton-X-100 at 37° C. for 45 min. Next, the macrophages were soaked in a blocking solution containing primary antibodies at 4° C. for 16 h. After rinsing with PBS, the macrophages were soaked in a blocking solution containing fluorophore-tagged secondary antibodies with phalloidin and DAPI at 25° C. for 45 min. After rinsing with PBS, the macrophages were mounted on a glass slide for imaging under LSM700 confocal microscope (Carl Zeiss) under identical conditions of laser exposure and image acquisition for comparing all the groups. Computations of the adherent macrophages were performed with ImageJ software. The number of DAPI-stained nuclei was counted from four different images to compute the macrophage adhesion density. The aspect ratio and spread area of the macrophages were analyzed by computing the major/minor axis and area of cells, respectively, from phalloidin-positive cell areas in four different images. The fluorescence intensity of protein expression was computed from the phalloidin-positive cell areas in four different images using a histogram function.

1. Experiments on Regulation of Macrophage Adhesion Depending on Degree of Ligand Dispersion

In order to examine whether the degree of macrophage adhesion can be regulated depending on the degree of ligand dispersion, whether proteins involved in macrophage polarization were expressed was tested using Examples 1 to 3 of the present invention, the case without the magnetic barriers and the ligands (Comparative Example 1), and the case without the ligands (Comparative Example 2). This experiment was conducted in a stationary state without applying a magnetic field.

a of FIG. 8 shows immunofluorescently stained images of F-actin, paxillin, and DAPI (nuclear staining) of macrophages after 24 h of culturing. Here, the expression of F-actin, paxillin and DAPI, which are cytoskeletal proteins, was clearly observed in Example 3 (low RGD dispersion condition). On the other hand, the expression of the proteins gradually decreased in Example 2 (moderate RGD dispersion condition) and Example 1 (high RGD dispersion condition). b of FIG. 8 depicts graphs showing the results of quantifying the experimental results, and it can be seen that, as the degree of RGD dispersion decreased, the adhesion density, the cell aspect ratio (e.g., elongated shape), and the cell area increased. This means that, even though the density of the blocked ligands is constant, when ligand dispersion is lowered, the adhesion complex of macrophages increases proportionally. a and b of FIG. 9 show the results of an experiment conducted to examine whether macrophages adhered to the nanobarrier under conditions excluding the magnetic barriers and the ligands or excluding only the ligands. The fluorescence images in a of FIG. 9 and the quantification graphs in b of FIG. 9 confirm that proteins were minimally expressed in all groups. These results clearly differ from the results in a and b of FIG. 8 obtained by conducting the experiment with the nanobarrier having both the magnetic barriers and the ligands. In the figures, the scale bar is 20 μm, and data are presented as mean±standard error (n=10). In the graphs, asterisks were assigned to p values with statistically significant differences (*:p<0.05; **: p<0.01; ***:p<0.001).

2. Experiment on Regulation of Macrophage Adhesion by Tuning of Nanogap

In order to examine whether macrophage adhesion is regulated depending on the nanogap size, an experiment was conducted by applying a magnetic field the nanobarriers of Experimental Examples 1 and 3. After 24 hours of cell culture, the expression levels of proteins were analyzed, and immunofluorescence imaging was performed in the same manner as 1 above. The lifting or dropping state was induced by applying a magnetic field to the nanobarriers. For comparison, a stationary state in which no magnetic field was applied was also observed.

a and b of FIG. 10 show experimental results obtained at high ligand dispersion, and c and d of FIG. 10 show experimental results obtained at low ligand dispersion. It can be seen that, in the high ligand dispersion condition, the adhesion of macrophages in the lifting state greatly increased compared to that in the stationary state, but the effect of inhibiting the adhesion of macrophages was small in the dropping state. In addition, it can be seen that, in the low ligand dispersion condition, the adhesion of macrophages in the dropping state greatly decreased compared to that in the stationary state, but the effect of facilitating macrophage adhesion was small in the lifting state. This suggests that, regardless of the degree of ligand dispersion, macrophage adhesion increases in the lifting state and is inhibited in the dropping state, but it is more effective to induce macrophage adhesion by inducing “lifting” in the high ligand dispersion condition and to inhibit macrophage adhesion by inducing “dropping” in the low ligand dispersion condition. In the figures, the scale bar is 20 μm, and data are presented as mean±standard error (n=10). In the graphs, asterisks were assigned to p values with statistically significant differences (*:p<0.05; **: p<0.01; ***:p<0.001).

3. Examination of Whether Integrins Adhere to Ligands

Whether integrins adhere to the unblocked ligands at the single cell level was examined by immunogold labeling and scanning electron microscope (SEM) images. After culturing macrophages, they were rinsed with 1,4 piperazine bis (2-ethanosulfonic acid) buffer (PIPES, pH=7.4, 0.1 M) for 2 min and fixed with 4% PFA for 12 min. After rinsing with PBS, the macrophages were permeabilized with Triton X-100 (0.5%) mixed with blocking buffer containing DI water, MgCl₂, NaCl, sucrose, and HEPES (pH=7.2) for 1 min. The treated macrophages were then placed in blocking buffer for 1 h and subsequently immersed in primary antibody (integrin β1, mouse) dissolved in blocking buffer at 37° C. for 2 h. After rinsing with 1% BSA, the MACROPHAGES were further blocked in a solution containing 5% goat serum for 12 min. The macrophages were subsequently incubated with secondary antibody-coated gold nanoparticles (AuNPs) in PIPES buffer for 16 h.

In this experiment, 40-nm-sized gold nanoparticles were used for immunolabeling of the adhered integrins such that they could be differentiated from 10 nm-sized AuNPs on the substrate surface. Secondary antibody-coated AuNPs were prepared by gently shaking 40-nm-sized AuNPs in secondary antibody [(goat anti-mouse) (H+L) IgG (Abcam)] in blocking buffer containing PIPES buffer (0.1 M, pH=7.4) mixed with 1% BSA and 0.1% Tween at 37° C. for 16 h. The immunolabeled integrins with 40 nm-sized AuNPs were rinsed with PIPES buffer and permanently fixed with 2.5% glutaraldehyde for 5 min. After rinsing THEM with PIPES buffer, they were further incubated in PIPES buffer mixed with 1% osmium tetroxide for 1 h to enhance the contrast in the SEM imaging of the macrophages. After serially rinsing with PIPES buffer and DI water and drying, they were subjected to SEM imaging. In the SEM images, cells were pseudo-colored in blue whereas the 40 nm AuNPs labeling the recruited integrin β1 were pseudo-colored in red. Using four different SEM images, the number of 40 nm AuNPs labeling the recruited integrin β1 per unit area was counted and then presented.

FIG. 11 shows that the lifting state effectively increased the adhesion of macrophages under the high ligand dispersion condition, and the dropping state effectively inhibited macrophage adhesion under the low ligand dispersion condition. In FIG. 11, the scale bar is 20 μm, and data are presented as mean±standard error (n=10). In the graphs, asterisks were assigned to p values with statistically significant differences (*:p<0.05; **: p<0.01; ***:p<0.001).

a to e of FIG. 12 show the results of an experiment conducted by labeling integrins with gold nanoparticles (40 nm) in order to investigate this effect in detail. c of FIG. 12 is a schematic view showing the experimental procedure in which integrins are labeled with gold nanoparticles (40 nm). In a and d of FIG. 12, it can be seen that, when the lifting state was formed by applying a magnetic field under the high ligand dispersion condition, the fluorescence intensity of integrins or the area of the adhered cells remarkably increased compared to that in the stationary state. In addition, it can be seen that, when the dropping state was formed by applying a magnetic field in the low ligand dispersion condition, the fluorescence intensity of integrins or the area of the adhered cells remarkably decreased compared to that in the stationary state. This can be confirmed more clearly with reference to b and e of FIG. depicting the results of quantifying the recruited integrins. The scale bar in a of FIG. 12 is 20 μm, and the scale bar in d of FIG. 12 is 200 nm. Data are presented as mean±standard error (n=10 in b of FIG. 10, and n=3 in d of FIG. 10). In the graphs, asterisks were assigned to p values with statistically significant differences (*:p<0.05; **: p<0.01; ***:p<0.001).

4. Experiment on Protein Expression in the Absence of Ligand

It was tested whether the adhesion and polarization of macrophages could be regulated by controlling only the state of the magnetic barriers in the absence of ligand. After excluding the ligand from the nanobarrier (Comparative Example 2) and applying a magnetic field to the magnetic barriers, the fluorescence intensities of proteins were measured.

a of FIG. 13 shows fluorescence images, and b of FIG. 13 depicts graphs showing the results of quantifying the density of adhered cells, the cell aspect ratio, and the cell area. It can be seen that proteins were hardly expressed in all cases regardless of conditions such as the size of the magnetic barriers, whether or not a magnetic field was applied, and the state of the magnetic barriers depending on the application of the magnetic field. This can be confirmed more clearly by referring to the quantification graph. Therefore, it can be confirmed that the presence of ligand is essential for regulating the adhesion of macrophages.

Summarizing the experiments of Experimental Example 3, it can be confirmed that macrophage integrins may adhere to the nanobarrier of the present invention and proteins may be expressed, regardless of the size of the magnetic barriers. However, it can be seen that, in order to regulate the adhesion of macrophages, the ligand dispersion condition may be modulated by modulating the size of the magnetic barriers, and the nanogap may be changed by application of a magnetic field, thereby effectively regulating the degree of adhesion of macrophages. In addition, it can be seen that the ligands, the magnetic barriers and the application of a magnetic field are essential for regulating macrophage adhesion.

### Experimental Example 4—Experiment on Regulation of Macrophage Adhesion and Polarization

1. Experiment on Analysis of Expression of Macrophage M1 and M2 Polarization Markers

It was tested whether the polarization direction of actual macrophages could be regulated by regulating the adhesion of macrophages through modulation of the nanobarrier. Here, the same method as in Experimental Example 3 was used for immunofluorescence staining.

Whether or not macrophage polarization was regulated was analyzed by Western blotting and quantification of proteins expressed in each polarization.

After culturing macrophages in an inflammation-inducing medium or an anti-inflammation-inducing medium for 36 hours while changing the degree of ligand dispersion and the height of the magnetic barriers, immunofluorescence staining images were taken.

Western blotting was performed as follows. After culturing macrophages in inflammation- or anti-inflammation-induction medium, they were lysed with PRO-PREP™ protein extraction buffer (iNtRON biotechnology, 400 μL) containing protease inhibitor cocktail (10 μL) for 20 min, and then centrifuged at 4° C. After centrifugation, the total protein concentration of the supernatant was quantified using a Thermo Scientific™ Pierce™ BCA Protein Assay Kit. The protein samples mixed with loading dye were subjected to denaturation by boiling at 100° C. for 8 min. 10% sodium dodecyl sulfate (SDS)-polyacrylamide gel electrophoresis (PAGE) was conducted at 110 V for 55 min to separate the denatured proteins. The samples were subsequently transferred to polyvinylidene fluoride (PVDF) membranes and further electrophoresed at 120 V for 90 min. Next, blocking buffer containing tris-buffered saline and 0.1% Tween 20 (TBST) mixed with 5% skimmed milk was used to block the transferred proteins at 4° C. for 16 h, followed by rinsing with TBST. The membranes were subsequently immersed in blocking buffer mixed with primary antibodies for iNOS (M1 polarization marker, 135 kDa), Arg-1 (M2 polarization marker, 37 kDa), and GAPDH (36 kDa) at 25° C. for 1 h. The membranes were thoroughly rinsed with TBST buffer and further incubated in the blocking buffer mixed with anti-horseradish peroxidase (HRP)-conjugated secondary antibodies at 25° C. for 1 h. Next, the membranes were rinsed with TBST buffer and treated with ECL western blotting reagent (Immobilon Western Chemiluminescent HRP Substrate, MERK-Millipore). The developed chemiluminescent signals in the PVDF membranes were imaged using a Linear Image Quant LAS 4000 mini chemiluminescent imaging system. The target protein expression levels were normalized to that of GAPDH and presented as the relative expression of target proteins.

a of FIG. 14 shows immunofluorescence staining images of each protein. It can be seen that, in under the high ligand dispersion condition, the expression of CD68, a marker of inflammatory polarization (M1 polarization), significantly decreased in the lifting state than in the stationary state. In addition, it can be seen that the expression of Arg-1, a marker of anti-inflammatory polarization (M2 polarization), significantly increased in the lifting state than in the stationary state.

Conversely, it can be seen that, in the low ligand dispersion condition, the expression of CD68, a marker protein of inflammatory polarization, significantly increased in the dropping state, and the expression of Arg-1, a marker protein of anti-inflammatory polarization, significantly decreased.

b of FIG. 14 (M1 induction medium) and c of FIG. 14 (M2 induction medium) are graphs showing the results of Western blotting and quantification of proteins after culturing macrophages for 36 hours while changing conditions. It can be seen that, in the inflammatory M1-induction medium and the high ligand dispersion condition, the expression of iNOS, a marker of inflammatory polarization (M1 polarization), significantly decreased in the lifting state than in the stationary state. On the other hand, it can be seen that, in the low ligand dispersion condition, the expression of iNOS significantly increased in the dropping state than in the stationary state. The same result can also be confirmed in the graph showing the results of quantifying the Western blot results. Arg-1 protein did not appear because M1 polarization-inducing medium was used.

Conversely, it can be seen that, in the anti-inflammatory M2-induction medium and the high ligand dispersion condition, the expression of Arg-1, a marker of anti-inflammatory polarization (M2 polarization), significantly increased in the lift state than in the stationary state. On the other hand, it can be seen that, in the low ligand dispersion condition, the expression of Arg-1, a marker protein of anti-inflammatory polarization, significantly decreased in the dropping state. The same result can also be confirmed in the graph showing the results of quantifying the Western blot results. iNOS protein did not appear because the M2 polarization-inducing medium was used. In the figures, the scale bar is 20 μm, and data are presented as mean±standard error (n=4). In the graphs, asterisks were assigned to p values with statistically significant differences (***: p<0.001).

2. Experiment after Addition of Macrophage Adhesion Protein Inhibitors

Next, in order to examine whether the regulation of polarization by macrophage adhesion is also associated with to the molecular machinery of cells, an experiment was condition after inhibitors of proteins involved in macrophage adhesion complex assembly were added. Since the formation of the lifting state in the high ligand dispersion condition significantly facilitated the adhesion-mediated M2 polarization of macrophages that could involve the formation of molecular machinery, the present inventors included this group. Contrastively, since the formation of the dropping state in the low ligand dispersion condition significantly suppressed the adhesion assembly in macrophages, the present inventors excluded this group.

FIG. 15 shows the results of examining the expression of ROCK2 in each condition before using the inhibitors. Referring to FIG. 15, it can be seen that both the group of the low ligand dispersion condition in the stationary state and the group of the high ligand dispersion condition in the lifting state effectively promoted the expression of ROCK2. In addition, in the high ligand dispersion condition, the anti-inflammatory polarization of macrophages in the lifting state was significantly promoted compared to that in the stationary state. The images in this figure are immunofluorescence images of ROCK2 and DAPI (nuclei) of adherent macrophages corresponding to the calculation of ROCK2 fluorescence intensity after 24 hours of macrophages in each condition. All experiments were repeated twice. The scale bar is 20 μm, and data are presented as mean±standard error (n=10). In the graph, asterisks were assigned to p values with statistically significant differences (**: p<0.01; ***: p<0.001).

a of FIG. 16 and FIG. 17 show the results of experiments conducted after inhibitors were added to inflammation-inducing medium in order to examine the relationship between the adhesion complex assembly and consequential polarization. As the inhibitors, inhibitors specific for myosin II (blebbistatin), actin polymerization (cytochalasin D), and ROCK (Y27632) were added. It was confirmed that, with high ligand dispersion in the stationary state, CD68 expression (implying the involvement of inflammation) was promoted with and without inhibitors. On the other hand, with low ligand dispersion in the stationary state and with high ligand dispersion in the lifting state, CD68 expression was promoted in all conditions with inhibitor, and CD68 expression was suppressed without inhibitors. All experiments were repeated twice. In the figures, the scale bar is 20 μm, and data are presented as mean±standard error (n=10). In the graphs, asterisks were assigned to p values with statistically significant differences (**: p<0.01; ***: p<0.001).

b of FIG. 16 and FIG. 18 show the results of experiments conducted after inhibitors were added to anti-inflammation-inducing medium in order to examine the relationship between the adhesion complex assembly and consequential polarization. The same inhibitors used for the inflammation-inducing medium were used. It can be confirmed that, with high dispersion in the stationary state, a low degree of anti-inflammatory Arg-1 expression was observed with and without inhibitors. Furthermore, all of the inhibitors were found to hinder Arg-1 expression with “low” ligand dispersion in the “stationary” state and “high” ligand dispersion in the “lifting” state, which was highly expressed in the absence of inhibitors. Here, all experiments were repeated twice. In the figures, the scale bar is 20 μm, and data are presented as mean±standard error (n=10). In the graphs, asterisks were assigned to p values with statistically significant differences (*: p<0.05; **: p<0.01; ***: p<0.001). ns means that the compared values are not statistically significantly different. These findings collectively prove that myosin II, actin polymerization, and ROCK function as the molecular mechanism with “low” ligand dispersion and contactless manipulation of RGD unclosing that augments the adhesion complex assembly-stimulated anti-inflammatory M2 polarization of macrophages while restraining inflammatory M1 polarization.

The results of this Experimental Example prove that it is possible to control the adhesion of macrophages by modulating the size of the magnetic barriers of the nanobarrier and tuning the nanogap, and as a result, the polarization direction of macrophages can be controlled. In addition, these results prove that it is possible to remotely regulate the adhesion and polarization of macrophages by the nanobarrier by applying a magnetic field to the nanobarrier.

### Experimental Example 5—Experiment on Regulation of Macrophage Adhesion and Polarization Using Nanobarrier In Vivo

It was tested whether the nanobarrier according to one embodiment of the present invention regulated the adhesion and polarization of macrophages even in vivo.

1. In Vivo Stabilization Test

The effect of contactless manipulation of the degree of ligand dispersion and the nanogap on regulating the polarization of host macrophages was analyzed via flow cytometry measurement. At 24 h post-implantation, recruited adherent host cells were collected via trypsinization and rinsed with ice-cold PBS mixed with 10% FBS. The host cells were collected via centrifugation, re-suspended in a fixing solution (4% PFA) for 10 min, and treated with blocking buffer containing PBS mixed with 3% BSA at 25° C. for 1 h. The blocked host cells were incubated in blocking buffer mixed with primary antibodies for M1 polarization-specific marker (iNOS) or M2 polarization-specific marker (Arg-1) at 25° C. for 1 h. A suitable isotype control antibody was also used. The host cells were rinsed with PBS and centrifuged, followed by incubating with fluorochrome-labeled secondary antibodies in buffer at 25° C. for 30 min in the dark. After rinsing the fluorochrome-labeled host cells with a solution containing PBS mixed with 3% BSA and 1% sodium azide, the fluorochrome-labeled host cells were analyzed via fluorescence-activated single cell sorting (FACS) Calibur and quantified via BD CellQuest Pro software (BD Biosciences). The analyzed data are presented as histograms using FlowJo software and the mean fluorescence intensities were quantified to their respective isotype control.

a of FIG. 19 schematically shows the process of subcutaneously implanting the nanobarrier into mice for this experiment. In a state in which no magnetic field was applied (stationary state), the magnet was in contact with the back of each mouse (inducing the lifting state) or in contact with the abdomen (inducing the dropping state). The implanted nanobarriers were collected 24 hours after implantation to examine the degree of degradation in vivo and confirm the effect of nanobarrier modulation.

a and b of FIG. 20 show the results of observing the nanobarrier before and 24 hours after implantation into mice. In a of FIG. 20, it can be seen that the shape and size of the nanobarrier before implantation into the mice were almost the same as after tuning the nanogap by applying a magnetic field for 24 hours after implantation into the mice. This indicates that the nanobarrier is stable in vivo and its stability does not change even when a magnetic field is applied thereto. This can also be confirmed in b of FIG. 20, which shows the results of quantifying the nanobarrier state before and after implantation. In the images, the scale bar is 200 nm, and the ligand-coated gold nanoparticles are marked in yellow and denoted as AuNPs. In the quantification graphs, data are presented as mean±standard error (n=20), and ns means that the compared values are not statistically significantly different.

2. Analysis of Regulation of Macrophage Polarization In Vivo

The effect on regulating the polarization of host macrophages recruited by modulating the nanobarrier under various conditions was analyzed via immunofluorescence staining images, flow cytometry quantification, and quantitative real time polymerase chain reaction (qPCR) analysis.

At 24 h post-implantation of the nanobarrier into mice, the recruited adherent host cells were lysed with Trizol (1 mL for each group) to extract RNA (900 ng per each group), which was subjected to reverse transcription using a High Capacity RNA-to-cDNA kit. The cDNA was amplified using Sybr Green assays and a StepOne Plus Real-Time PCR System (Applied Biosystems) via real-time PCR cycles. The relative expressions of target genes [M1 polarization marker (iNOS) and M2 polarization marker (Arg-1)] were normalized to that of the housekeeping gene (GAPDH) and displayed.

As shown in b of FIG. 19, a and b of FIG. 20, and FIG. 21, it can be confirmed that, with the “high” ligand dispersion, the adhesion complex (F-actin assembly) and Arg-1 were significantly intensified while iNOS expression was substantially suppressed in the “lifting” state compared to the “stationary” state. Conversely, with “low” ligand dispersion, the adhesion complex and Arg-1 expression were significantly suppressed while iNOS expression was considerably stimulated in the “dropping” state compared to the “stationary” state. Indeed, flow cytometry histograms with quantifications and qPCR-based relative gene expression levels of host cells consistently corroborated these trends (c of FIG. 19). Similarly, these analysis results suggest that the “dropping” condition in the low ligand dispersion switched pronounced Arg-1 expression to marked iNOS expression.

While the present invention has been described with reference to the particular illustrative embodiments, it will be understood by those skilled in the art to which the present invention pertains that the present invention may be embodied in other specific forms without departing from the technical spirit or essential characteristics of the present invention. Therefore, the embodiments described above are considered to be illustrative in all respects and not restrictive. Furthermore, the scope of the present invention is defined by the appended claims rather than the detailed description, and it should be understood that all modifications or variations derived from the meanings and scope of the present invention and equivalents thereto are included in the scope of the present invention.

What is claimed is:

1. A nanobarrier for regulating macrophage adhesion and polarization comprising:
magnetic barriers each comprising an aggregate of one or more magnetic particle units;
a linker connected to one side of each of the magnetic barriers; and
a substrate connected to the magnetic barriers via the linkers,
wherein the substrate comprises ligands to which macrophages adhere.

2. The nanobarrier of claim 1, wherein an average diameter of the magnetic barriers includes any one or more of a first average diameter, a second average diameter, and a third average diameter,
wherein the first average diameter is 150 to 250 nm, the second average diameter is 450 to 530 nm, and the third average diameter is 650 to 750 nm.

3. The nanobarrier of claim 2, wherein a surface of each magnetic barrier, which faces the substrate, is spaced apart from each ligand present on the substrate by a distance of a nanogap,
wherein the nanogap is reversibly changed by application of a magnetic field.

4. The nanobarrier of claim 3, wherein the average diameter of the magnetic barriers includes the first average diameter, and macrophage M2 polarization is promoted by elongating the linker and increasing the nanogap, through pulling of the magnetic barriers in a direction away from the substrate by application of the magnetic field.

5. The nanobarrier of claim 3, wherein the average diameter of the magnetic barriers includes the third average diameter, and macrophage M1 polarization is promoted by compressing the linker and reducing the nanogap, through pulling of the magnetic barriers in a direction toward the substrate by application of the magnetic field.

6. The nanobarrier of claim 1, wherein the linker comprises: a polyethylene glycol (PEG) portion; a first bonding portion which forms a chemical bond with the magnetic barrier; and a second bonding portion which forms a chemical bond with the substrate.

7. The nanobarrier of claim 1, wherein
the magnetic barrier includes a carboxylate group (—COO⁻),
the first bonding portion includes any one of an amino group (—NH₂) and a thiol group (—SH) and forms a chemical bond with the carboxylate group of the magnetic barrier, and
the second bonding portion includes any one of a maleimide group and an alkenyl group (—C═C—) and form a chemical bond with a thiol group (—SH) provided on the substrate.

8. The nanobarrier of claim 1, wherein the linker has a structure of the following Formula 1: 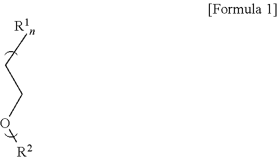
wherein n is 30 to 5,000, R¹ is any one of an amino group (—NH₂) and a thiol group (—SH), and R² is any one of a maleimide group and an alkenyl group (—C═C—).

9. The nanobarrier of claim 1, wherein the linker has a length of 10 nm to 1 μm.

10. The nanobarrier of claim 1, wherein the ligands provided on the substrate are bound to surfaces of gold nanoparticles bound to the substrate.

11. The nanobarrier of claim 10, wherein
the gold nanoparticles are provided on the substrate by chemical bonding with a portion of the thiol groups (—SH) provided on the substrate,
the ligands are bound to the gold nanoparticles, and
the linkers are connected to the substrate by chemical bonding with the other portion of the thiol groups (—SH) provided on the substrate.

12. The nanobarrier of claim 10, wherein the gold nanoparticles cover 0.001% to 10% of the area of the substrate.

13. The nanobarrier of claim 1, wherein 70 to 85% of the area of the substrate is covered by the magnetic barriers.

14. The nanobarrier of claim 1, wherein the nanobarrier is prepared by:
forming aggregates of one or more magnetic particle units;
forming a carboxylate group on the surfaces of the aggregates to form magnetic barriers;
binding each of the magnetic barriers to one end of each linker by stirring the magnetic barriers and the linkers;
chemically binding the other end of each linker to thiol groups on a substrate on which thiol groups and ligands are present; and
deactivating thiol groups on the substrate, which remain unbound to the linkers.

15. The nanobarrier of claim 14, wherein
the substrate comprises a glass substrate, and thiol groups and ligands provided on at least one surface of the glass substrate,
the thiol groups are provided by thiolizing the glass substrate,
at least a portion of the thiol groups are bound to gold nanoparticles, and
the ligands are bound to the gold nanoparticles bound to the thiol groups.

16. A method of regulating macrophage adhesion and polarization using a nanobarrier, the method comprising regulating macrophage adhesion and polarization by applying a magnetic field to the nanobarrier according to claim 1.

17. The method of claim 16, wherein the magnetic field is applied from outside the body to remotely control the nanobarrier in the body.

18. The method of claim 16, wherein the magnetic field has a strength of 100 mT to 500 mT.

19. The method of claim 16, wherein the magnetic barriers are pulled in a direction away from the substrate by the magnetic field to elongate the linker, thereby inhibiting macrophage M1 polarization and promoting macrophage M2 polarization.

20. The method of claim 16, wherein the magnetic barriers are pulled in a direction toward the substrate by the magnetic field to compress the linker, thereby inhibiting macrophage M2 polarization and promoting macrophage M1 polarization.
